(19) United States
(12) Patent Application Publication
(10) Pub. No.: US 2024/0071581 A1
(43) Pub. Date: Feb. 29, 2024

(54) METHOD FOR MANAGING CHART USING SPEECH RECOGNITION AND DEVICE USING SAME

(71) Applicants: Byung Joon LIM, Seoul (KR); DENCOMM INC., Seoul (KR)

(72) Inventor: Byung Joon LIM, Seoul (KR)

(21) Appl. No.: 18/280,311

(22) PCT Filed: Mar. 9, 2021
(86) PCT No.: PCT/KR2021/002917

§ 371 Date: Sep. 5, 2023

(30) Foreign Application Priority Data
Mar. 8, 2021 (KR) 10-2021-0030408

(51) Int. Cl. G16H 10/60; G10L 15/26

(52) U.S. Cl. CPC G16H 10/60; G10L 15/26

(57) ABSTRACT

A method for managing a chart may include recognizing speech of a user and generating same as speech data, converting the speech data to text data, extracting a system command and/or a data command from the text data, on the basis of the system command, performing starting a customer chart, ending the customer chart, and/or moving to an item included in the customer chart, extracting a diagnosis name command and a status command by parsing the data command, generating a protocol by mapping the diagnosis name command and the status command, on the basis of the protocol, changing the status of at least one of the plurality of items in the customer chart, and providing the user, in real time, with the status of an item and the changed items in the customer chart.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2021/002917, filed Mar. 9, 2021, which claims priority to the benefit of Korean Patent Application No. 10-2021-0030408 filed in the Korean Intellectual Property Office on Mar. 8, 2021, the entire contents of which are incorporated herein by reference.

### BACKGROUND

### 1. Technical Field

The present invention relates to a chart management method using speech recognition and a device using the same, and more particularly, to a chart management method and a device using the same, wherein a user's speech is recognized in real time, commands are classified and identified for inputting status values into a specific area of the chart, and the status values which are the diagnosis results are automatically input to the chart using the identified commands, thereby effectively managing the chart.

### 2. Background Art

In general, dental treatment is made in a manner in which a doctor checks the condition of a patient's teeth with the naked eyes and provides diagnosis information in technical terms, and an assistant (e.g., a nurse) next to the doctor writes down the diagnosis information by hand in the patient's medical chart.

However, if the assistant does not properly understand or misunderstands the doctor's terms, the patient's dental condition may be incorrectly recorded in the chart or important information may be omitted, which can lead to medical accidents.

Therefore, to accurately understand and record the diagnosis made by the doctor, professional personnel who have undergone specialized training courses are needed. However, in a dental clinic where it is difficult to recruit the professional personnel, the doctor has to perform various processes alone, such as checking the condition of the teeth, documenting a chart, and performing treatment, making it difficult to focus only on the treatment.

In addition, when a plurality of assistants supports the doctor, it may be difficult to divide duties smoothly, to secure the doctor's field of view, or to prevent their movement from overlapping, which interferes with the medical treatment.

### SUMMARY

The inventive concept of the present invention relates to providing a chart management method using speech recognition and a device using the same, capable of recognizing a user's speech, converting the speech data into text data, extracting meaningful system commands and data commands from the text data, and automatically inputting status values of various items constituting a customer chart.

The inventive concept of the present invention is not limited to the mentioned above, and other inventive concepts not mentioned will be clearly understood by those skilled in the art from the following description.

The inventive concept provides a chart management method using speech recognition according to an embodiment, the method including (a) recognizing a user's speech and acquiring speech data using a speech input unit, (b) converting the speech data into text data using a speech recognition unit, (c) extracting at least one of a system command and a data command from the text data using a controller, (d) performing at least one of starting a customer chart, ending the customer chart, and moving to at least one of a plurality of items included in the customer chart based on the system command using the controller, (e) parsing the data command using the controller to extract a diagnosis name command and a status command, (f) generating a protocol by mapping the diagnosis name command to the status command using the controller, (g) changing a status of at least one of the plurality of items in the customer chart based on the protocol using the controller, and (h) providing the user with the status of at least one of the plurality of items in the customer chart and the plurality of changed items in real time using a display unit. Accordingly, it is possible for the user to conveniently and effectively document the customer charts using speech recognition without manual documentation.

Details of other embodiments are included in the detailed description and drawings.

According to the present invention as described above, it has various effects as follows.

The present invention which recognizes a user's speech and automatically reflects the recognized diagnosis to a customer chart using speech recognition has an effect of reducing the number of assistants required to record the user's diagnosis information on the chart and reducing the risks of omission and incorrect entries that may occur while writing down the information by hand.

The purpose and various advantages of the present invention will become more apparent from preferred embodiments of the present invention by those skilled in the art.

### BRIEF DESCRIPTION OF THE DRAWINGS

FIG. 1 is a block diagram showing a schematic configuration of a chart management device using speech recognition according to an embodiment of the present invention.

FIG. 2 is a flowchart illustrating a chart management method using speech recognition according to an embodiment of the present invention.

### DETAILED DESCRIPTION

Hereinafter, various embodiments of the present invention will be described in detail with reference to the accompanying drawings. The advantages and features of the present invention, and methods for the same, will become clear with reference to the embodiments described below in detail in conjunction with the accompanying drawings. However, the present invention is not limited to the embodiments disclosed below, but will be implemented in a variety of different forms. The embodiments are provided only to make the disclosure of the present invention complete, and to fully inform those skilled in the art of the scope of the invention to which the present invention belongs, and the present invention is defined by the scope of the claims.

The shapes, sizes, ratios, angles, numbers, and the like disclosed in the drawings for explaining the embodiments of the present invention are illustrative, so the present invention is not limited to the illustrated details. Throughout the specification, the same reference numerals refer to the same components. In addition, in explaining the present invention, when it is determined that a detailed description of related known technologies may unnecessarily obscure the subject matter of the present invention, the detailed description will be omitted. When terms such as “include”, “consist of”, and the like mentioned in this specification are used, other parts may be added unless “only” is used. When the number of components is expressed as a singular, the present invention includes a case in which a plurality of components is included unless otherwise explicitly stated.

In interpreting the components, it is interpreted that an error range is included even without a separate explicit description.

In describing a positional relationship, for example, when the positional relationship of two parts is described with “on”, “above”, “below”, “next to”, and the like, one or more other parts may be located between the two parts unless “immediately” or “directly” is used.

When an element or a layer is referred to as being “on” another element or another layer, it includes all cases where an element or a layer is directly on top of another element or another layer or where the other element or the other layer are interposed therebetween.

Although first, second, and the like are used to describe various components, these components are not limited by these terms. These terms are only used to distinguish one component from another. Therefore, a first component mentioned below may also be a second component within the technical spirit of the present invention.

Throughout the specification, the same reference numerals refer to the same components.

The size and thickness of each component shown in the drawings are illustrated for convenience of description, and the present invention is not necessarily limited to the size and thickness of the illustrated components.

The features of the various embodiments of the present invention may be partially or wholly combined with each other and are capable of various interlocking and driving as those skilled in the art can fully understand, and the embodiments may be implemented independently or together in an associative relationship.

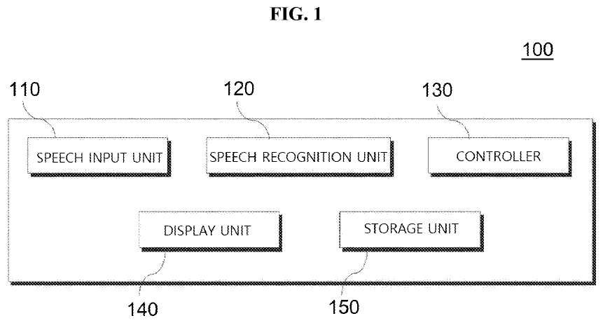

FIG. 1 is a block diagram showing a schematic configuration of a chart management device using speech recognition according to an embodiment of the present invention.

Referring to FIG. 1, the chart management device 100 using speech recognition according to the present invention may include a speech input unit 110, a speech recognition unit 120, a controller 130, a display unit 140, and a storage unit 150.

The speech input unit 110 of the chart management device 100 is configured to receive a user's speech and acquire speech data. The speech input unit 110 may receive a user's speech like a microphone or hands-free, and the user may place the speech input unit 110 around the mouth or wear the speech input unit 110 on the body. Accordingly, when dealing with a customer, the user may use both hands freely without holding the speech input unit 110 with his or her hand. For example, the user may be a dentist, and various dental treatment tools, such as a dental mirror, explorer, tweezers, suction, light, handpiece, and the like, may be used with both hands, as the user mounts or wears the speech input unit 110 on clothes or the body. The user who uses the chart management device 100 of the present invention is not limited to a dentist as in the above example, and various users may use the chart management device 100 of the present invention as needed.

The speech recognition unit 120 of the chart management device 100 is configured to convert the speech data into text data. The speech recognition unit 120 may receive data from the speech input unit 110 and convert the speech data into text data by analyzing a waveform of the speech data. The speech recognition unit 120 may analyze the speech data received from the speech input unit 110 in real time, and may transmit the text data generated through the analysis to the controller 130 in real time.

The speech recognition unit 120 may use artificial intelligence that is learned using deep learning or machine learning as a speech recognition algorithm.

The controller 130 of the chart management device 100 is configured to extract various commands from the text data and change status values of a plurality of items of a customer chart based on the commands. Specifically, the controller 130 may receive the text data from the speech recognition unit 120 and extract a plurality of commands from the text data. The plurality of commands may be commands capable of creating a customer chart, selecting at least one of the plurality of items constituting the customer chart, and changing a status value of the selected item.

The controller 130 of the chart management device 100 may create a customer chart. The customer chart may be a table with rows and columns. The customer chart may include the customer number of the corresponding customer, the chart number of the corresponding chart, and the diagnosis result for each of a plurality of teeth. For example, multiple columns of the table of the customer chart may be divided based on tooth numbers. The diagnosis information about tooth number 1 may be described in some columns, and the diagnosis information about tooth number 2 may be described in other columns adjacent to the columns of tooth number 1. As such, a plurality of rows of the table may be configured according to a plurality of tooth numbers. In a plurality of rows of the table of the customer chart, diagnosis names for each tooth may be recorded. That is, when one row corresponding to the diagnosis name and one column corresponding to the tooth number are selected, one cell of the table may be designated, and a diagnosis result value may be input to the corresponding cell.

More specifically, the plurality of commands may include system commands and data commands. The system commands may be commands capable of controlling the entire system of the chart management device 100. By using the system commands, the user may create a new customer chart, start or end the customer chart, input a customer number or a chart number, move to each of a plurality of items constituting the customer chart, cancel or edit the input data, and reset all the input data.

For example, the customer chart may be a customer chart for a patient receiving treatment at a dental clinic. In this case, the system commands may include at least one of a start command, an end command, a customer number command, a chart number command, a tooth number command, a next tooth command, a previous tooth command, a tooth front command, a tooth back command, an input undo command, and a data reset command.

More specifically, the start command is a command for starting the documentation of the customer chart, the end command is a command for ending the documentation of the customer chart, the customer number command is a command for starting the input of a customer number which is the customer's unique number, the chart number command is a command for starting the input of the chart number unique to the customer chart, the tooth number command is a command for selecting one of unique tooth numbers of each of a plurality of teeth of the customer, the next tooth command is a command for selecting a tooth with a number following the tooth number of the tooth for which the status value is currently being input, the previous tooth command is a command for selecting a tooth with a number previous to the tooth number of the tooth for which the current status value is being input, the tooth front command is a command for selecting the front surface of a tooth to input a status value for the front surface of the tooth for which the status value is currently being input, the tooth back command is a command for selecting the back side of a tooth to input a status value for the back side of the tooth for which the current status value is being input, and the data reset command is a command for deleting and resetting all status values in the entire customer chart, all status values for the tooth of the currently input tooth number, or all status values for the input item of the currently input tooth.

Among the plurality of commands, the data commands may be commands for selecting each of the plurality of items constituting the customer chart and changing the status value of the selected item. In the case of the customer chart for a patient undergoing dental treatment as mentioned above, a plurality of teeth may be classified based on tooth numbers, and each of the plurality of teeth may have a plurality of items. The plurality of items may correspond to various diagnoses of the tooth. That is, tooth number 1 may be diagnosed based on the plurality of items, and the plurality of items may include the degree of gum recession, the degree of gingival margin, the presence or absence of plague, the degree of exposure of root bifurcation, the presence or absence of crown, the presence or absence of bridge, the presence or absence of implant, and the degree of shaking. Each of the plurality of teeth may be diagnosed for each of the plurality of items, and a status value, which is a result value, may be input to the diagnosis corresponding to each of the plurality of items.

More specifically, the data commands may include diagnosis name commands and status commands. The diagnosis name command is a command corresponding to each of the plurality of items, and one of the plurality of items in the customer chart may be selected based on the diagnosis name command. That is, the diagnosis name command may correspond to a diagnosis name when diagnosing a corresponding tooth.

As such, the diagnosis name command may refer to a detailed diagnosis name for a customer's specific tooth, i.e., a specific tooth corresponding to the selected tooth number, selected through the system command. For example, the diagnosis name command may include at least one of a gum recession command, a gingival margin command, a plague command, a bridge command, an implant command, and a shaking command.

More specifically, among the diagnosis name commands, the gum recession command is a command for selecting a corresponding item to input a status value for the degree of gum recession of the corresponding tooth, the gingival margin command is a command for selecting a corresponding item to input a status value for the gingival margin, the plague command is a command for selecting a corresponding item to input a status value for the presence or absence of plague, a bleeding status command is a command for selecting a corresponding item to input a status value for the presence or absence of bleeding in the corresponding tooth, a root bifurcation command is a command for selecting a corresponding item to input a status value for the root bifurcation, the crown command is a command for selecting a corresponding item to input a status value for the presence or absence of a crown, the bridge command is a command for selecting a corresponding item to input a status value for the presence or existence of a bridge, the implant command is a command for selecting a corresponding item to input a status value for the presence or absence of an implant, and the shaking command is a command for selecting a corresponding item to input a status value for the degree of shaking of the corresponding tooth.

In addition, the status command which is a command for a status value for a plurality of items may correspond to a diagnosis result value for a diagnosis name corresponding to the diagnosis name command. Based on the status command, the status value of the selected item may be input, edited, or deleted.

More specifically, the status command corresponding to the gum recession command is a status value (1 to 10) for the degree of gum recession of the corresponding tooth, the status command corresponding to the gingival margin command is a status value (0 to 3) for the gingival margin, the status command corresponding to the plague command is a status value (YES/NO) for the presence or absence of plague, the status command corresponding to the bleeding status command is a status value (YES/NO) for the presence or absence of bleeding in the corresponding tooth, the status command corresponding to the root bifurcation command is a status value (1 to 3) for the root bifurcation, the status command corresponding to the crown command is a status value (YES/NO) for the presence or absence of a crown, the status command corresponding to the bridge command is a status value (YES/NO) for the presence or absence of a bridge, the status command corresponding to the implant command is a status value (YES/NO) for the presence or absence of an implant, and the status command corresponding to the shaking command may be a status value (1 to 3) for the degree of shaking of the corresponding tooth.

In addition, the diagnosis name command and the status command may be mapped to each other by the controller 130. The diagnosis name command and the status command may correspond to each other, and may be mapped to generate a protocol when they correspond to each other. The controller 130 may map the diagnosis name command to the status command according to various criteria. For example, the controller 130 may map the diagnosis name command to the status command based on the proximity and sequence of the diagnostic name command and the status command. When the recognition time of a specific diagnosis name command and the recognition time of a status command are equal to or less than a preset time, it may be determined that the corresponding diagnosis name command and status command are close enough to be mapped. In addition, in the order in which the diagnosis name command and the status command are recognized by the controller 130, the diagnosis name command must be recognized prior to the status command so that they can be mapped to each other. That is, when the diagnosis name command is first recognized and the status command is recognized later before a preset time elapses, the controller 130 may map the diagnosis name command to the status command.

The controller 130 may generate one protocol by mapping the diagnosis name command to the status command, and based on this protocol, select one of the plurality of items in the customer chart and change the status value of the corresponding item.

The display unit 140 of the chart management device 100 is configured to display the created customer chart and display the plurality of items in the customer chart changed by the controller 130 in real time. As described above, using the text data transferred from the speech recognition unit 120 to the controller 130, the controller 130 may recognize a plurality of commands, may select an item to be changed in the customer chart by the system command, the diagnosis name command, and the status command, and may change the status value of the corresponding item. The change made by the controller 130 may be displayed on the display unit 140, and the user may check the changed status value of the item in real time.

The storage unit 150 of the chart management device 100 may be a database that stores information on a plurality of commands used by the controller 130 and information on customer charts. The storage unit 150 may store the system commands and the data commands, such as diagnosis name commands and status commands. In addition, the storage unit 150 may store information about the customer chart that is changed, e.g., created, input, and edited, by the controller 130.

Subsequently, a chart management method of the chart management device 100 using speech recognition will be described in more detail.

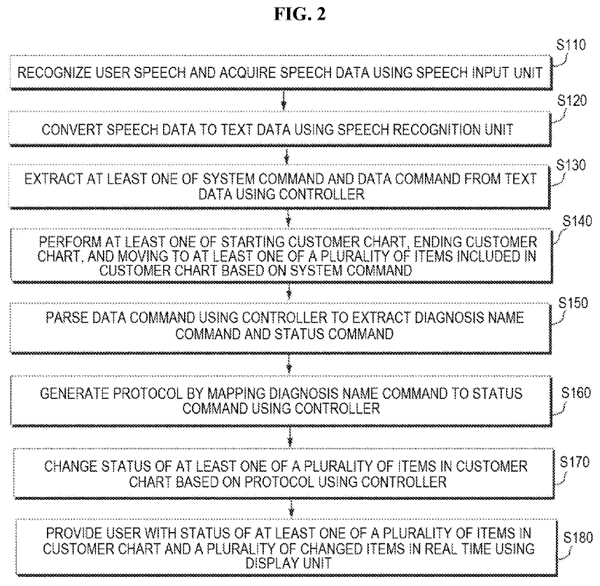

FIG. 2 is a flowchart illustrating a chart management method using speech recognition according to an embodiment of the present invention.

First, a user's speech may be recognized and acquired as speech data using the speech input unit 110 (S110). The speech input unit 110 may receive the user's speech and acquire speech data. The speech input unit 110 may transmit the acquired speech data to the speech recognition unit 120 in real time.

Subsequently, the speech data may be converted into text data using the speech recognition unit 120 (S120). The speech recognition unit 120 may convert the received speech data into text data in real time. The speech recognition unit 120 may transmit the converted text data to the controller 130 in real time.

Subsequently, at least one of a system command and a data command may be extracted from the text data using the controller 130 (S130). The controller 130 may parse the text data received in real time and extract a plurality of commands that are meaningful data. As described above, the commands may include the system command and the data command, and the controller 130 may parse the text data to extract the system command and the data command.

Subsequently, at least one of starting a customer chart, ending the customer chart, and moving to at least one of a plurality of items included in the customer chart may be performed based on the system command using the controller 130 (S140). The controller 130 may recognize the extracted system command and change the operation of the chart management system in response to the recognized system command.

For example, the controller 130 may recognize a start command among system commands. The user may say, for example, “START” as the start command, and the controller 130 may create a new customer chart or retrieve an existing customer chart to start documentation of the customer chart based on the speech-recognized start command.

In addition, the controller 130 may recognize a customer number command and a chart number command among the system commands. The user may say “CUSTOMER NUMBER 12345” as the customer number command, and the controller 130 may input or change the customer number of the created customer chart to 12345 based on the speech-recognized customer number command. In addition, the user may say “CHART NUMBER 54321” as the chart number command, and the controller 130 may input or change the chart number of the created customer chart to 54321 based on the speech-recognized chart number command.

In addition, the controller 130 may recognize a tooth number command among system commands. The user may say “TOOTH NUMBER 3” as the tooth number command, and the controller 130 may move to an area corresponding to the tooth with tooth number 3 among a plurality of teeth in the customer chart based on the speech-recognized tooth number command.

In addition, the controller 130 may recognize a next tooth command or a previous tooth command among system commands. When the user says “NEXT TOOTH” as the next tooth command or “PREVIOUS TOOTH” as the previous tooth command, the controller 130 may recognize the command and move from the current area corresponding to the tooth with tooth number 3 to the area corresponding to the next tooth, tooth number 4, or to the area corresponding to the previous tooth, tooth number 2.

In addition, when recognizing “TOOTH FRONT” or “TOOTH BACK” as the tooth front command or the tooth back command among system commands, the controller 130 may move from the current area corresponding to the tooth with tooth number 3 to the area corresponding to the front side of the tooth with tooth number 3 or the area corresponding to the back side of the tooth with tooth number 3.

Subsequently, the diagnosis name command and the status command may be extracted by parsing the data command using the controller 130 (S150). Once recognizing the system command using the text data, the controller 130 may move to an area in the customer chart that needs to be changed, such as input and edit, and then extract the data command to change the status value of a plurality of items in the area.

As described above, the controller 130 may extract the diagnosis name command and status command by parsing the data command. For example, the user may say “PLAGUE YES” and the controller 130 may recognize “PLAGUE” in the text data as the plague command among the diagnosis name commands and “YES” as the status command through speech recognition.

Subsequently, a protocol may be generated by mapping the diagnosis name command to the status command using the controller 130 (S160). The controller 130 may generate a protocol by mapping the recognized plague command to the status command when they satisfy a preset condition. As described above, if “YES” is later recognized before a preset time elapses after “PLAGUE”, which is text data, is first recognized, the controller 130 may map the plague command recognized through “PLAGUE” to the status command recognized through “YES” to generate one protocol.

Subsequently, the status of at least one of a plurality of items in the customer chart may be changed based on the protocol using the controller 130 (S170). The controller 130 has moved to a specific area in the customer chart in step S140, and may move to, for example, an area corresponding to the front surface of the tooth with tooth number 3 among a plurality of teeth having different tooth numbers. The front surface of the tooth with tooth number 3 may have a plurality of items for diagnosing the front surface of the tooth with tooth number 3. The controller 130 may change the status value of at least one of the plurality of items corresponding to the front surface of the tooth with tooth number 3 using a protocol generated through the data commands, that is, the diagnosis name command and the status command in step S160. If the controller 130 recognizes “PLAGUE” and “YES” as data commands as in the example above, the controller 130 may newly input a status value of “YES” to an item corresponding to the presence or absence of plague as diagnosis information on the front surface of the tooth with tooth number 3. Accordingly, data indicating that plague is present on the front surface of the tooth with tooth number 3 of the customer may be input to the corresponding customer chart.

Subsequently, the status of at least one of the plurality of items in the customer chart and the plurality of changed items may be provided to the user in real time using the display unit 140 (S180). When the controller 130 creates the customer chart and moves to various areas of the customer chart, the display unit 140 may display changes in the changed items among the plurality of items in real time. For example, when the controller 130 moves to a specific area of the customer chart in step S140, for example, to the area corresponding to the front surface of the tooth with tooth number 3, the display unit 140 may display an area corresponding to the front surface of the tooth with tooth number 3 while displaying the entire screen of the customer chart. In addition, when the controller 130 changes the status of at least one of the plurality of items in step S170, the display unit 140 may display the status value of the changed item in real time. Since the display unit 140 displays the plurality of items and displays the status of the plurality of changed items in real time, the user may check in real time whether speech recognition is being performed correctly and may check the changed status value in real time.

According to an embodiment, a chart management method using speech recognition and a device using the same may recognize a user's speech to extract a plurality of commands, analyze the plurality of commands to change the contents in the customer chart in real time, and display the changes on screen for provision to the user. Therefore, the user may not directly write down information about the customer, or a third party adjacent to the user may not write down information about the result of diagnosis stated by the user. That is, the user may input desired information into the customer chart by using speech recognition, and both hands may be free while managing the customer chart. Therefore, it is possible to more conveniently and usefully manage, e.g., input or edit, customer charts.

According to another embodiment of the present invention, a chart management method using speech recognition and a device using the same are substantially the same as the chart management method and the device using the same of FIGS. 1 and 2, except that the following steps are further included, and thus redundant descriptions are omitted. For convenience of explanation, it will be described with reference to FIGS. 1 and 2.

In the chart management method using speech recognition, when an error occurs in extracting the system command and the data command in step S130, or when an error occurs in extracting the diagnosis name command and status command in step S150, a confirmation request message may be delivered to the user by using the controller.

Specifically, the controller extracts the system command and the data command from the text data in step S130, and various errors may occur during the extraction process. In this case, the controller may recognize that an error has occurred in extracting the commands from the text data, and may deliver a notification message using the display unit to re-input commands due to the error. Accordingly, the user may recognize that the speech input spoken by the user has not been properly recognized by the controller, and may input speech to the speech input unit once again.

In addition, an error may occur while the controller extracts the diagnosis name command and status command in step S150. For example, the controller may determine that the extracted diagnostic name command and the status command do not correspond to each other, and may determine that a protocol is generated by mapping the diagnostic name command to the status command. For example, when the user may say “CROWN 10”, and the controller may extract a crown command (diagnosis name command) which is “CROWN” and a status command which is “10” by using text data converted from the speech data. However, the status command corresponding to the crown command may be YES or NO as a status value for the presence or absence of the crown, and may be stored in the storage unit of the chart management device and retrieved by the controller. The controller may confirm that the status command corresponding to the crown command is YES or NO, and may determine that the extracted status command “10” does not correspond to the crown command. Accordingly, the controller may determine that the crown command and the status command of “10” cannot be mapped to each other to generate a protocol. In this case, the display unit may display a confirmation request message to prompt input of a status command corresponding to the recognized crown command. Accordingly, the user may provide a new speech input including a correct status command corresponding to the crown command.

For another example, the controller may determine that the extracted diagnostic name command and the status command may correspond to each other but are a combination of commands that are extracted at a low frequency. For example, in determining that the commands are a combination of commands that are extracted at a low frequency, the user may provide a speech input of “GUM RECESSION 10”, and the controller may extract a gum recession command (diagnosis name command) of “GUM RECESSION” and a status command of “10” by using text data converted from the speech data. The status command corresponding to the gum recession command may have a value ranging from 1 to 10 as a status value for the degree of gum recession. Accordingly, the controller may determine that the gum recession command and the status command of “10” may correspond to each other. However, the controller may retrieve the frequency of the status value of the status command corresponding to the gum recession using the status commands corresponding to the gum recession command in customer charts of the plurality of customers stored in the storage unit. When the status value corresponding to the gum recession command is 3 to 6, it may be frequent, and when the status value corresponding to the gum recession command is 10, it may not be frequent. The controller may determine that “10” recognized as the status command may be an error when cases in which the status command corresponding to the gum recession command is 10 are less than or equal to a preset frequency. Accordingly, the display unit may display a confirmation request message to ask the user to confirm whether the degree of gum recession is “10”. Accordingly, the user may input a speech signal indicating that it was a correct input, or may provide a new speech input if the previous input of “10” was an incorrect input.

According to an embodiment, the chart management device using speech recognition may further include a speech output unit. The speech output unit may be configured to output speech, like a speaker. The speech output unit may transmit a message to the user by outputting speech. Therefore, the speech output unit may transmit the confirmation request message to the user by speech in the above situation, and may more effectively deliver the message to the user other than the display unit.

According to another embodiment of the present invention, a chart management method using speech recognition and a device using the same are substantially the same as the chart management method and the device using the same of FIGS. 1 and 2, except that the following steps are further included, and thus redundant descriptions are omitted. For convenience of explanation, it will be described with reference to FIGS. 1 and 2.

In the chart management method using speech recognition, one of a plurality of grades may be assigned based on the status of a plurality of items of a customer chart by using the controller. The chart management device may perform steps S110 to S180 to assign a grade to each of a plurality of teeth of the customer based on the completed customer chart, and may assign a grade to the customer based on the grade assigned to each of the plurality of teeth.

Specifically, one of the plurality of grades may be assigned to each of the plurality of teeth of the corresponding customer based on the period remaining until treatment is to begin. The period remaining until treatment is to begin may include a year, six months, a month, and immediately. The shorter the period remaining until treatment is to begin, the higher the grade may be assigned. For example, if there is no time left until treatment is to begin and treatment must begin immediately, the highest grade may be assigned, and if there is one month left until treatment is to begin, a lower grade may be assigned than the case in which treatment must begin immediately. For example, the highest grade, grade 1, may be assigned a tooth that needs to be treated immediately, and grade 2, which is lower than grade 1, may be assigned when the period remaining until treatment is to begin is a month.

In addition, a grade corresponding to the highest grade among grades assigned to a plurality of teeth of a specific customer may be assigned to the customer. For example, for the specific customer, grade 2 may be assigned to a tooth with tooth number 3 and grade number 4 may be assigned to a tooth with tooth number 5 among the plurality of teeth. In this case, grade 2, which is the highest grade among grades 2 and 4, may be assigned to the specific customer.

When a grade corresponding to the highest grade among grades assigned to a plurality of teeth of a customer may be assigned to the customer, it is possible to effectively manage the customer. Since the tooth with the shortest period remaining until treatment is to begin needs to be treated first among the plurality of teeth of the customer, treatment may be preferentially offered to the customer with a high grade by assigning the high grade to the customer having the tooth with the high grade.

Subsequently, a notification message may be delivered to the customer of the customer chart based on one of the plurality of grades using the controller. The chart management device may deliver a notification message suggesting treatment based on grades assigned to a plurality of teeth of the specific customer. Specifically, the chart management device may determine when to deliver the notification message in response to the grade assigned to the tooth, and may deliver the notification message accordingly. If some of the plurality of teeth of the specific customer have grade 1, a notification message may be delivered to the customer who has finished treatment to notify that the corresponding teeth need to be treated immediately. In addition, when some of the other teeth of the plurality of teeth of the specific customer have grade 2, a notification message may be delivered to the customer who has finished treatment to notify that the corresponding teeth need to be treated in one month after the date of treatment.

According to an embodiment, when the grades assigned to the plurality of teeth of the customer is changed, the grade assigned to the customer may be changed correspondingly. If the tooth to which the highest grade (e.g., grade 2) is assigned among the plurality of teeth of the specific customer is treated, the grade (grade 2) assigned to the tooth that has been treated may be removed, and the highest grade may be changed to another grade (e.g., grade 3). Accordingly, the customer's grade may be changed to grade 3.

According to another embodiment, a chart management method using speech recognition and a device using the same are substantially the same as the chart management method and the device using the same of FIGS. 1 and 2, except that the following steps are further included, and thus redundant descriptions are omitted. For convenience of explanation, it will be described with reference to FIGS. 1 and 2.

The chart management method using speech recognition measures at least one of the customer's hand grip pressure and vital sign using a customer state measuring unit, and may deliver a notification message to the user based on at least one of the pressure and the vital sign.

Specifically, the chart management device may further include a customer state measuring unit. The customer state measuring unit is configured to measure the state of the customer, such as the degree of tension and the degree of feeling pain. The customer state measuring unit may include at least one of a pressure measuring unit and a vital sign measuring unit.

The pressure measuring unit may be configured to measure the hand grip pressure, that is, the grip strength. The customer may hold the pressure measuring unit in his or her hand, and the customer's hand grip pressure on the pressure measuring unit may be measured according to the degree of fear or pain while the dental diagnosis and treatment are in progress. The pressure measuring unit may transmit the measured customer's grip strength to the controller in real time. The controller may deliver a notification message to the user when the pressure gripped by the hand is greater than or equal to a preset pressure. Accordingly, the user may check the customer's state and adjust the degree of diagnosis and treatment.

The vital sign measuring unit is configured to measure at least one of the customer's body temperature and heart rate. The vital sign measuring unit may be attached to the body of the customer, measure the change in the customer's body temperature or heart rate in real time, and transmit it to the controller. The controller may compare the customer's body temperature with a preset body temperature, and deliver a notification message to the user when a difference between the reference body temperature and the customer's body temperature is greater than or equal to a preset difference. In addition, the controller may deliver a notification message to the user when the customer's heart rate decreases below a preset heart rate. Accordingly, the user may check the customer's state and adjust the degree of diagnosis and treatment.

According to an embodiment, the controller of the chart management device may receive information on the gum depth and thickness from a probe used by the user in real time, and may reflect the information on the customer's chart in real time. Specifically, the probe is a device having a bent end to measure the depth of inflammation of periodontal disease, and is inserted into the customer's gums by the user to automatically measure the depth of the gums.

One or more of the various embodiments of the present invention may be combined to form a new embodiment.

According to various embodiments of the present invention, a chart management method using speech recognition includes (a) recognizing a user's speech and acquiring speech data using the speech input unit, (b) converting the speech data into text data using a speech recognition unit, (c) extracting at least one of a system command and a data command from the text data using a controller, (d) performing at least one of starting a customer chart, ending the customer chart, and moving to at least one of a plurality of items included in the customer chart based on the system command by using the controller, (e) parsing the data command using the controller to extract a diagnosis name command and a status command, (f) generating a protocol by mapping the diagnosis name command to the status command using the controller, (g) changing a status of at least one of the plurality of items in the customer chart based on the protocol using the controller, and (h) providing the user with the status of at least one of the plurality of items in the customer chart and the plurality of changed items in real time using a display unit.

According to another feature of the present invention, a diagnosis name command among data commands may correspond to at least one of a plurality of items in the customer chart, and a status command among data commands may correspond to a status value of at least one of a plurality of items.

According to another feature of the present invention, in step (f), mapping may be performed based on the proximity and sequence of the diagnostic name command and the status command.

According to another feature of the present invention, the system commands include at least one of a start command, an end command, a customer number command, a chart number command, a tooth number command, a next tooth command, a previous tooth command, a tooth front command, a tooth back command, an input undo command, and a data reset command.

According to another feature of the present invention, the diagnosis name commands may include at least one of a gum recession command, a gingival margin command, a plague command, a bleeding status command, a root bifurcation command, a crown command, a bridge command, an implant command, and a shaking command.

According to another feature of the present invention, if an error occurs in extracting the diagnosis name command and the status command in step (c), or if an error occurs in extracting the diagnosis name command and status command in step (e), the method may further include transmitting a confirmation request message to the user using the controller.

According to another feature of the present invention, the method may further include assigning one of a plurality of grades based on the status of the plurality of items in the customer chart using the controller, and delivering a notification message to the customer in the customer chart based on one of the plurality of grades.

According to another feature of the present invention, the method may further include transmitting a notification message to the user based on at least one of the customer's hand grip pressure and vital sign, using the customer state measuring unit.

According to various embodiments of the present invention, a chart management device using speech recognition includes a speech input unit configured to recognize a user's speech and acquire speech data, a speech recognition unit configured to convert the speech data into text data, a controller configured to extract at least one of a system command and a data command from the text data, perform at least one of starting a customer chart, ending the customer chart, and moving to at least one of a plurality of items included in the customer chart based on the system command, parse the data command to extract a diagnosis name command and a status command, generate a protocol by mapping the diagnosis name command to the status command, and change the status of at least one of the plurality of items in the customer chart based on the protocol, and a display unit configured to provide the user with the status of at least one of the plurality of items in the customer chart and the changed plurality of items in real time.

It should be understood that embodiments described herein should be considered in a descriptive sense only and not for purposes of limitation. Descriptions of features or aspects within each embodiment should typically be considered as available for other similar features or aspects in other embodiments.

Therefore, it will be understood by those of ordinary skill in the art that various changes in form and details may be made therein without departing from the spirit and scope of the disclosure as defined by the following claims.

1. A method for managing a chart using speech recognition, the method comprising:
(a) recognizing a user's speech and acquiring speech data using a speech input unit;
(b) converting the speech data into text data using a speech recognition unit;
(c) extracting at least one of a system command and a data command from the text data using a controller;
(d) performing at least one of starting a customer chart, ending the customer chart, and moving to at least one of a plurality of items included in the customer chart based on the system command by using the controller;
(e) parsing the data command using the controller to extract a diagnosis name command and a status command;
(f) generating a protocol by mapping the diagnosis name command to the status command using the controller;
(g) changing a status of at least one of the plurality of items in the customer chart based on the protocol using the controller; and
(h) providing the user with the status of at least one of the plurality of items in the customer chart and the plurality of changed items in real time using a display unit.

2. The method of claim 1, wherein the diagnosis name command among the data commands corresponds to at least one of the plurality of items in the customer chart, and the status command among the data commands corresponds to at least one status value among the plurality of items.

3. The method of claim 1, wherein the generating of the protocol includes mapping the diagnosis name command to the status command based on the proximity and sequence thereof.

4. The method of claim 1, wherein the system command includes at least one of a start command, an end command, a customer number command, a chart number command, a tooth number command, a next tooth command, a previous tooth command, a tooth front command, a tooth back command, an input undo command, and a data reset command.

5. The method of claim 1, wherein the diagnosis name command includes at least one of a gum recession command, a gingival margin command, a plague command, a bleeding status command, a root bifurcation command, a crown command, a bridge command, an implant command, and a shaking command.

6. The method of claim 1, further comprising transmitting a confirmation request message to the user using the controller when an error occurs in extracting the system command and the data command in the extracting of the at least one of the system command and the data command, or when an error occurs in extracting the diagnosis name command and the status command in the extracting of the at least one of the system command and the data command.

7. The method of claim 1, further comprising assigning one of a plurality of grades based on the status of the plurality of items in the customer chart by using the controller; and delivering a notification message to the customer of the customer chart based on one of the plurality of grades.

8. The method of claim 1, further comprising measuring at least one of a customer's hand grip pressure and vital sign using a customer state measuring unit, and transmitting a notification message to the user based on at least one of the pressure and the vital sign.

9. A device for managing a chart using speech recognition, the device comprising:
a speech input unit configured to recognize a user's speech and acquire speech data;
a speech recognition unit configured to convert the speech data into text data;
a controller configured to extract at least one of a system command and a data command from the text data,
perform at least one of starting a customer chart, ending the customer chart, and moving to at least one of a plurality of items included in the customer chart based on the system command,
parse the data command to extract a diagnosis name command and a status command,
generate a protocol by mapping the diagnosis name command to the status command, and
change the status of at least one of the plurality of items in the customer chart based on the protocol; and
a display unit configured to provide the user with the status of at least one of the plurality of items in the customer chart and the changed plurality of items in real time.
